(19) United States
(12) Patent Application Publication
(10) Pub. No.: US 2020/0268141 A1
(43) Pub. Date: Aug. 27, 2020

(54) Voice Assistant in an Electric Toothbrush

(71) Applicant: The Procter & Gamble Company, Cincinnati, OH (US)

(72) Inventors: Matthew Lloyd Newman, Cincinnati, OH (US); Patrick M Schwing, Indian Hill, OH (US); Peter Charles Mason, Jr., South Lebanon, OH (US)

(21) Appl. No.: 16/803,137

(22) Filed: Feb. 27, 2020

Related U.S. Application Data
(60) Provisional application No. 62/811,086, filed on Feb. 27, 2019.

(51) Int. Cl. A46B 15/00; A46B 9/04; A46B 13/02; A46B 5/00; A46B 7/04; G09B 19/00; G10L 15/19; G10L 15/22; H04R 1/02; G10L 25/51; G06F 3/16; A61C 17/22

(52) U.S. Cl. CPC A46B 15/0028; A61C 17/221; A46B 9/04; A46B 15/0006; A46B 15/001; A46B 15/0012; A46B 15/0022; A46B 13/02; A46B 5/0095; A46B 7/04; G09B 19/0084; A46B 15/0095; G10L 15/19; G10L 15/22; H04R 1/028; G10L 25/51; G06F 3/165; G06F 3/167; A61C 17/224; G10L 2015/223; A46B 15/004

(57) ABSTRACT

A voice-activated electric toothbrush system including an electric toothbrush, a charging station such as an inductive charging station that provides power to the electric toothbrush, and a voice-assistant application that may be included in the electric toothbrush or the charging station. The device that includes the voice-assistant application may also include one or more microphones for receiving voice input, such as a microphone array, and one or more speakers for providing voice output, such as a speaker array. The toothbrush and the charging station may communicate with each other via a short-range communication link—and may also communicate with a client computing device of the user via short-range communication. The electric toothbrush may include one or more sensors for detecting sensor data during a brushing session which may be used when generating the voice output.

### TECHNICAL FIELD

The present disclosure generally relates to electric toothbrush systems, and, more particularly, to a voice assistant for receiving voice input and providing voice output at an electric toothbrush.

### BACKGROUND

Typically, an electric toothbrush has a toothbrush head and a toothbrush handle. The electric toothbrush receives power from an inductive charging station by coupling the electric toothbrush to the inductive charging station. Users control the electric toothbrush via buttons and switches on the electric toothbrush handle. However, users typically are not made aware of their brushing habits, such as the average length of time in which they brush their teeth, whether they are using the appropriate amount of force, areas they may have missed when brushing, etc. Furthermore, users do not know when the electric toothbrush needs to be charged or when the toothbrush head needs to be changed. Moreover, electric toothbrushes do not have a mechanism for users to communicate with the electric toothbrush to receive any of this information.

### SUMMARY

To communicate with and control an electric toothbrush, the electric toothbrush includes a voice assistant that receives voice input from a user, analyzes the voice input to identify a request from the user, determines an action to perform based on the request, and provides a voice response to the user or controls operation of the electric toothbrush based on the request. For example, the user may request to turn on the electric toothbrush by saying, “Toothbrush on.” In response to the request, the voice assistant may transmit a control signal to the electric toothbrush handle to turn the power on. In some scenarios, the voice assistant provides voice output without a request from the user. For example, the voice assistant may continuously or periodically determine the battery life remaining for the electric toothbrush—and may generate an announcement to the user to charge the electric toothbrush when the battery life remaining is less than a threshold battery percentage. Additionally, the voice assistant may continuously or periodically estimate the life remaining for the electric toothbrush head—and may generate an announcement to the user to change the electric toothbrush head when the estimated life remaining is less than a threshold number of brushing sessions.

In this manner, the electric toothbrush may communicate directly with the user during a brushing session to improve the user's brushing performance. The user does not have to stop brushing and look at a separate device to see the areas in which she needs to improve her brushing habits or to see segments which could use additional attention before she finishes brushing. Through the voice assistant, the electric toothbrush may interact with the user in real-time to provide the optimal brushing experience.

In some embodiments, the voice assistant is included in a charging station that provides power to the electric toothbrush. More specifically, the charging station may be an inductive charging station and may include one or more microphones to receive voice input, one or more speakers to provide voice output, and one or more processors that execute instructions stored in a memory. The instructions may cause the processors to recognize speech, determine requests, identify actions to perform based on the requests, and provide voice output or control operation of the electric toothbrush based on the requests. The charging station may also include a communication interface to communicate with the electric toothbrush and/or a client computing device of the user via a short-range communication link. The communication interface may also be used to communicate with remote servers via a long-range communication link, such as the Internet.

In this manner, the charging station may communicate with remote servers, such as a natural language processing server, to determine the request based on voice input from the user. The charging station may also communicate with the electric toothbrush to send control signals to the electric toothbrush and to receive sensor data from the electric toothbrush for generating the voice output. For example, the charging station may receive sensor data from the electric toothbrush to identify segments of the user's teeth that the user has not brushed or has not brushed thoroughly. Then the charging station may provide a voice instruction to the user to brush the identified segments. Additionally, the charging station may communicate with the user's client computing device to provide user performance data for presentation and storage by an electric toothbrush application executing on the user's client computing device.

In one embodiment, a system for providing voice assistance regarding an electric toothbrush includes an electric toothbrush, and a charging station configured to provide power to the electric toothbrush. The charging station includes a communication interface, one or more processors, a speaker, a microphone, and a non-transitory computer-readable memory coupled to the one or more processors, the speaker, the microphone, and the communication interface, and storing instructions thereon. The instructions, when executed by the one or more processors, cause the charging station to receive, from a user via the microphone, voice input regarding the electric toothbrush, and provide, to the user via the speaker, voice output related to the electric toothbrush.

In another embodiment, a method for providing voice assistance regarding an electric toothbrush includes receiving, at a charging station providing power to an electric toothbrush, voice input via a microphone from a user of the electric toothbrush. The method further includes analyzing the received voice input to determine a request from the user, determining an action in response to the request, and performing an action in response to the request by providing, via a speaker, a voice response to the request, providing a visual indicator, or adjusting operation of the electric toothbrush based on the request.

In yet another embodiment, a method for providing voice assistance regarding an electric toothbrush includes during a brushing session by a user, obtaining, at a charging station providing power to an electric toothbrush, sensor data from one or more sensors included in the electric toothbrush. The method further includes analyzing the sensor data to identify one or more user performance metrics related to use of the electric toothbrush by the user, and providing, via a speaker, voice output to the user based on the one or more user performance metrics.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment of thereof. Further, wherever possible, the following description refers to the reference numerals included in the following figures, in which features depicted in multiple figures are designated with consistent reference numerals.

FIG. 1 illustrates an example voice-activated electric toothbrush system having an electric toothbrush and a charging station with a voice assistant;

FIG. 2 illustrates an example electric toothbrush having an electric toothbrush handle and an electric toothbrush head that can operate in the system of FIG. 1;

FIG. 3 illustrates a block diagram of an example communication system in which the electric toothbrush and the charging station can operate;

FIG. 4 illustrates example voice inputs that may be provided to the voice assistant, and example requests and actions for the voice assistant to perform based on the received voice inputs;

FIG. 5 illustrates example actions that the voice assistant may perform, and example voice outputs that the voice assistant may provide based on the actions;

FIG. 6 illustrates a flow diagram of an example method for providing voice assistance to a user regarding an electric toothbrush, which can be implemented in the charging station; and

FIG. 7 illustrates a flow diagram of another example method for providing voice assistance to a user regarding an electric toothbrush, which can be implemented in the charging station.

### DETAILED DESCRIPTION

Although the following text sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments could be implemented, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112(f).

Generally speaking, techniques for providing voice assistance regarding an electric toothbrush may be implemented in an electric toothbrush, in a charging station that provides power to the electric toothbrush, in one or more network servers such as a natural language processing server or an action determination server, in one or more client computing devices, and/or a system that includes several of these devices. However, for clarity, the examples below focus primarily on an embodiment in which a charging station that includes voice assistance functionality receives voice input from a user. The charging station transcribes the voice input to text input and provides the text input or the raw voice input to a natural language processing server to identify a request based on the voice input. The charging station receives the identified request and provides the identified request to an action determination server that identifies an action for the charging station to perform based on the request and one or more steps to complete the action. Then the charging station receives the identified action and performs each of the steps.

In some scenarios, one of the steps may include receiving sensor data from the electric toothbrush. In other scenarios, one of the steps may include receiving data from the user's client computing device. Also in some scenarios, a step may include providing voice output to the user responding to the request, providing a visual indicator such as light from a light emitting diode (LED) to the user responding to the request, or sending a control signal to the electric toothbrush to control/adjust operation of the electric toothbrush based on the request. The visual indicator may be used to indicate for example, that the electric toothbrush has been turned on or turned off in response to a request by the user to turn on or turn off electric toothbrush. The charging station may also provide data, such as user performance data indicative of the user's brushing behavior to the client computing device for presentation or storage at an electric toothbrush application executing on the client computing device.

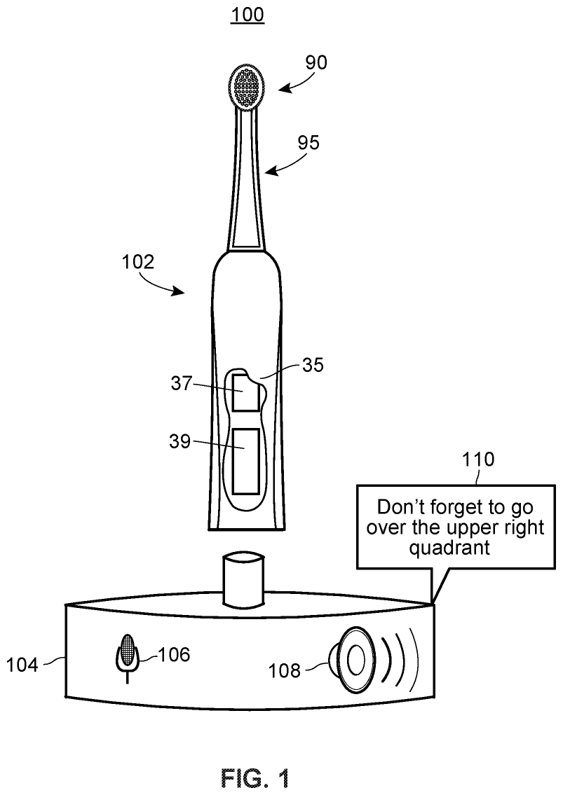

FIG. 1 illustrates various aspects of an exemplary environment implementing a voice-activated electric toothbrush system 100. The voice-activated electric toothbrush system 100 includes an electric toothbrush 102 and a charging station 104 such as an inductive charging station that provides power to the electric toothbrush 102 when the electric toothbrush is coupled to the charging station 104. The charging station 104, described in more detail below, includes a voice assistant having one or more microphones 106, such as an array of microphones 106 and one or more speakers 108, such as an array of speakers 108. The voice assistant may also include processors and a memory storing instructions for receiving and analyzing voice input and providing voice output 110, such as “Don't forget to go over the upper right quadrant.” The voice assistant included in the charging station 104 may include the hardware and software components of the voice controlled assistant described in U.S. Pat. No. 9,304,736 filed on Apr. 18, 2013, incorporated by reference herein.

The electric toothbrush 102 may include a motor 37 and an energy source 39 that is in electrical communication with the motor 37. The motor is operatively coupled to one or more movable bristle holders disposed on the head 90 to move one or more of the bristle holders. The bristles holders can rotate, oscillate, translate, vibrate, or undergo a movement that is a combination thereof. The head 90 can be provided as a removable head so that it can be removed and replaced when the bristles (or other components) of the bristle holder have deteriorated. Examples of electric toothbrushes that may be used with the present invention, including examples of drive systems for operatively coupling the motor to the bristle holders (or otherwise moving the one or more bristle holders or the head), types of cleaning elements for use on a bristle holder, structures suitable for use with removable heads, bristle holder movements, other structural components and features, and operational or functional features or characteristics of electric toothbrushes are disclosed in USPNs 2002/0129454; 2005/0000044; 2003/0101526; U.S. Pat. Nos. 5,577,285; 5,311,633; 5,289,604; 5,974,615; 5,930,858; 5,943,723; 2003/0154567; 2003/0163881; 2005/0235439; U.S. Pat. No. 6,648,641; 2005/0050658; 2005/0050659; 2005/0053895; 2005/0066459; 2004/0154112; U.S. Pat. No. 6,058,541; and PCT/US2005/008050 (U.S. Pat. No. 8,214,958).

The electric toothbrush 102 may also include an electric toothbrush handle 35 and an electric toothbrush head 90 removably attached to the electric toothbrush handle 35 and having a neck 95. In some embodiments, the electric toothbrush may include one or more sensors which may be included in the head 90, neck 95, or handle 35 of the electric toothbrush. The sensors may include light or imaging sensors such as cameras, electromagnetic field sensors such as Hall sensors, capacitance sensors, resistance sensors, inductive sensors, humidity sensors, movement or acceleration or inclination sensors such as multi-axis accelerometers, pressure sensors, gas sensors, vibration sensors, temperature sensors, or any other suitable sensors for detecting characteristics of the electric toothbrush 102 or of the user's brushing performance with the electric toothbrush 102. Also in some embodiments, the electric toothbrush 102 may include one or more LEDs, for example on the electric toothbrush handle 35. The LEDs may be used to indicate whether the electric toothbrush 102 is turned on or turned off, the mode for the electric toothbrush 102, such as daily clean, massage or gum care, sensitive, whitening, deep clean, or tongue clean, the brush speed or frequency for the electric toothbrush head 90, etc. In other embodiments, the LEDs may be included on the charging station 104.

In any event, the charging station 104 can be used to recharge the power source, such as a battery, within the electric toothbrush 102. The charging station 104 can be configured to receive a plurality of electric toothbrushes, or other oral-care products such as manual toothbrushes, accessories for the electric toothbrush 102 (such as a plurality of heads or other attachments), and/or other personal-care products. The charging station can be coupled by a power cord to an external source of power, such as an AC outlet (not shown).

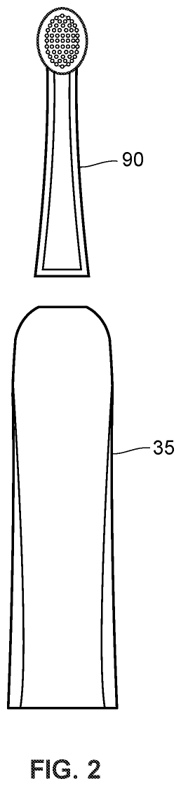

As mentioned above, the electric toothbrush 102 may include an electric toothbrush handle 35 and an electric toothbrush head 90 that is removably attached to the electric toothbrush handle 35 as shown in FIG. 2. In some embodiments, the electric toothbrush head 90 is disposable and several electric toothbrush heads 90 may be attached to and removed from the electric toothbrush handle 35. For example, a family of four may share the same electric toothbrush handle 35 while each attaching their own electric toothbrush head 90 to the electric toothbrush handle 35 during use. Additionally, the electric toothbrush heads 90 may have limited lifespans, and a user may change out an old electric toothbrush head for a new electric toothbrush head after a certain number of uses.

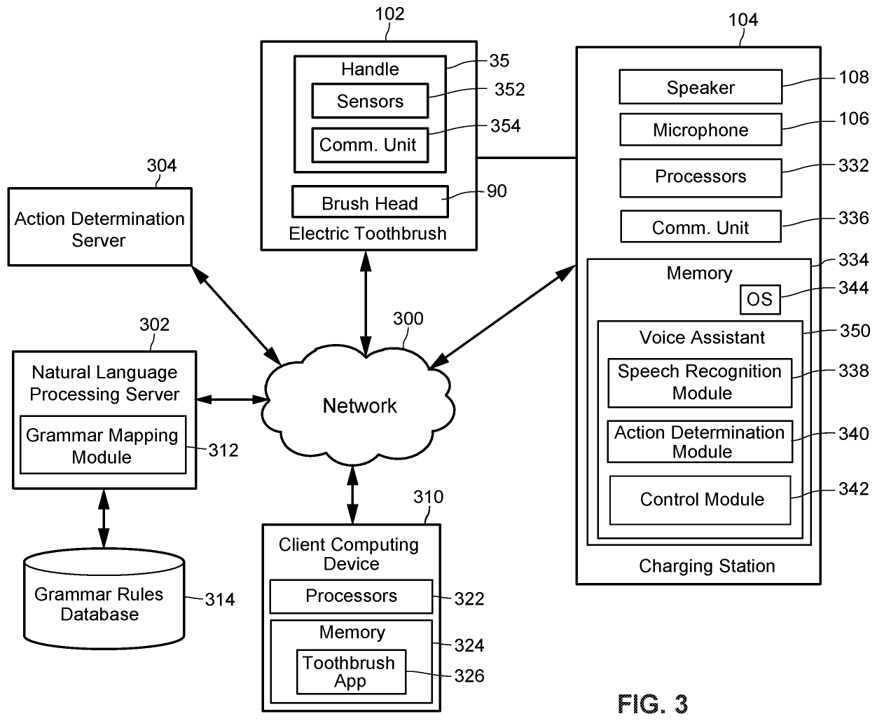

FIG. 3 illustrates an example communication system which the electric toothbrush 102 and the charging station 104 can operate to provide voice assistance. The electric toothbrush 102 and the charging station 104 have access to a wide area communication network 300 such as the Internet via a long-range wireless communication link (e.g., a cellular link). In the example configuration of FIG. 3, the electric toothbrush 102 and the charging station 104 communicate with a natural language processing server 302 that converts voice instructions to requests in which the devices can respond, and an action determination server 304 that identifies an action for the charging station 104 to perform in response to the request and one or more steps for the charging station 104 to perform to carry out the action. More generally, the electric toothbrush 102, and the charging station 104 can communicate with any number of suitable servers.

The electric toothbrush 102 and the charging station 104 can also use a variety of arrangements, singly or in combination, to communicate with each other and/or with a client computing device 310 of the user, such as a tablet or smartphone. In some embodiments, the electric toothbrush 102, the charging station 104, and the client computing device 310 communicate over a short-range communication link, such as short-range radio frequency links including Bluetooth™, Wi-Fi (802.11 based or the like) or another type of radio frequency link, such as wireless USB. In other embodiments, the short-range communication link may be an infrared (IR) communication link using for example, an IR wavelength of 950 nm modulated at 36 KHz.

As shown in FIG. 3, the charging station 104 may include one or more speakers 108 such as an array of speakers, one or more microphones 106 such as an array of microphones, one or more processors 332, a communication unit 336 to transmit and receive data over long-range and short-range communication networks, and a memory 334.

The memory 334 can store instructions of an operating system 344 and a voice assistant application 350. The voice assistant application 350 may receive voice input and/or provide voice output, provide a visual indicator, or control operations of the electric toothbrush 102 via a speech recognition module 338, an action determination module 340, and a control module 342. While the voice assistant application 350 is shown as being stored in the memory 334 of the charging station 104, this is merely one example embodiment for ease of illustration only. In other embodiments, the voice assistant application 350, the one or more speakers 108, and the one or more microphones 106 may be included in the electric toothbrush 102.

In any event, the voice assistant application 350 may receive voice input from a user, and the speech recognition module 338 may transcribe the voice input to text using speech recognition techniques. In some embodiments, the speech recognition module 338 may transmit the voice input to a remote server such as a speech recognition server, and may receive corresponding text transcribed by the speech recognition server. The text may then be compared to grammar rules stored at the charging station 104, or may be transmitted to the natural language processing server 302. For example, the charging station 104 or the natural language processing server 302 may store a list of candidate requests that the voice assistant application 350 can handle, such as turning on and off the electric toothbrush, and selecting the brushing mode for the electric toothbrush, such as daily clean, massage or gum care, sensitive, whitening, deep clean, or tongue clean. The requests may also include identifying the amount of charge or battery life remaining for the electric toothbrush 102, identifying the number of brushing sessions remaining before the electric toothbrush requires additional charge, identifying the life remaining for the brush head, identifying user performance metrics for the current brushing session or previous brushing sessions, sending user performance data to the user's client computing device, etc. However, a user may intend the same request by using a wide variety of voice input. For example, to request the electric toothbrush 102 to change the brushing mode to the sensitive mode, the user may say, “Sensitive mode,” “Set mode to sensitive,” “Gentle mode,” “Brush softer,” etc. The speech recognition module 338 may include a set of grammar rules for receiving voice input or voice input transcribed to text and determining a request from the voice input.

The action determination module 340 may then identify an action based on the determined request and one or more steps for carrying out the action. For example, when the request is to turn off the electric toothbrush 102, the action determination module 340 may identify the action as turning off the power for the electric toothbrush 102 and the one or more steps for carrying out the action as sending a control signal to the electric toothbrush 102 to turn off the power.

In another example, when the request is to determine segments of the user's teeth which require additional attention, the action determination module 340 may identify the action as providing a voice response indicating the segments which require additional attention. The one or more steps for carrying out the action may include obtaining historical user performance data for the user to identify segments which have not been brushed as thoroughly as other segments in the past. The historical user performance data may be obtained from the user's client computing device 310, from the action determination server 304, or from a toothbrush server which communicates with the toothbrush application 326 stored on the user's client computing device 310. The one or more steps may also include obtaining sensor data from the electric toothbrush 102 and analyzing the sensor data to identify segments which have not been brushed as thoroughly as other segments in the current brushing session.

More specifically, the electric toothbrush 102 may periodically or continuously provide sensor data in real-time or at least near real-time for the current brushing session to the charging station 104 via a short-range communication link. The sensor data may include data indicating the positions of the electric toothbrush 102 at several instances in time, for example from multi-axis accelerometers and/or cameras included in the electric toothbrush 102. The sensor data may also include data indicating the amount of force exerted by the user at several instances in time, for example from pressure sensors included in the electric toothbrush 102. The action determination module 340 may analyze the positions at several instances in time to identify movement of the electric toothbrush 102 and the amount of force exerted at each position to identify segments of the user's teeth which have not been brushed at all, and to identify the proportion of the total surface area that has been brushed in a segment.

For example, the user's teeth may be divided into four segments: the upper left quadrant of the user's teeth, the upper right quadrant, the lower left quadrant, and the lower right quadrant. Based on the detected positions of the electric toothbrush 102 at several instances in time and the amount of force exerted at each position, the action determination module 340 may determine that the user has not brushed the upper right quadrant. Accordingly, the action determination module 340 may generate a voice response to the user to brush the upper right quadrant. In another example, based on the detected positions of the electric toothbrush 102 at several instances in time and the amount of force exerted at each position, the action determination module 340 may determine that the user has brushed 50 percent of the total surface area of the lower left quadrant. The proportion of the total surface area that has been brushed in a segment may be compared to a threshold amount (e.g., 90 percent). If the proportion is less than the threshold amount, the action determination module 340 may generate a voice response to go over the lower left quadrant.

In other examples, the user's teeth may be divided into 12 segments: the inner surface of the upper left quadrant, the outer surface of the upper left quadrant, the chewing surface of the upper left quadrant, the inner surface of the upper right quadrant, the outer surface of the upper right quadrant, the chewing surface of the upper right quadrant, the inner surface of the lower left quadrant, the outer surface of the lower left quadrant, the chewing surface of the lower left quadrant, the inner surface of the lower right quadrant, the outer surface of the lower right quadrant, and the chewing surface of the lower right quadrant.

In some embodiments, the action determination module 340 may transmit the request to a remote server such as the action determination server 304, and may receive a corresponding action and one or more steps to carry out the action from the action determination server 304. The action determination module 340 may then perform the one or more steps. Also in some embodiments, the action determination module 340 may communicate with the control module 342 to carry out the action. The control module 342 may control operation of the electric toothbrush 102 by transmitting control signals to the electric toothbrush 102 via the short-range communication link. The control signals may cause the electric toothbrush 102 to turn on, turn off, change the brushing mode to a particular brushing mode, change the brush speed or frequency, etc. When the action involves controlling operation of the electric toothbrush 102, the action determination module 340 may provide a request to the control module 342 to provide the corresponding control signals for the electric toothbrush 102 to perform a particular operation.

As described above, the electric toothbrush 102 may include an electric toothbrush handle 35 and an electric toothbrush head 90 removably attached to the handle 35. The handle 35 may further include one or more sensors 352 and a communication unit 354 for communicating with the charging station 104 and/or the client computing device 10 over a network via short-range communication links and/or remote servers via a long-range communication link 300. The one or more sensors 352 may include light or imaging sensors such as cameras, electromagnetic field sensors such as Hall sensors, capacitance sensors, resistance sensors, inductive sensors, humidity sensors, movement or acceleration or inclination sensors such as multi-axis accelerometers, pressure sensors, gas sensors, vibration sensors, temperature sensors, or any other suitable sensors for detecting characteristics of the electric toothbrush 102 or of the user's brushing performance with the electric toothbrush 102. While the one or more sensors 352 are shown in FIG. 3 as being included in the handle 35, the one or more sensors 352 may be included in the head 90, or may be included in a combination of the head 90 and the handle 35.

The natural language processing server 302 may receive text transcribed from voice input from the charging station 104. For example, the charging station 104 may transcribe the voice input to text via the speech recognition module 338 included in the voice assistant application 350. A grammar mapping module 312 within the natural language processing server 302 may then compare the received text corresponding to the voice input to grammar rules in a grammar rules database 314. For example, based on the grammar rules, the grammar mapping module 312 may determine for the input, “Toothbrush on,” that the request is to turn on the electric toothbrush 102.

Moreover, the grammar mapping module 112 may make inferences based on context. For example, a voice input may be for user performance data right after a brushing session, but the user may not specify whether the user performance data should be for the most recent brushing session or historical brushing sessions. However, the grammar mapping module 312 may infer that the request is for user performance data for the most recent brushing session, for example, using machine learning. In another example, when the voice input is for user performance data and the user has not brushed her teeth within a threshold amount of time, grammar mapping module 312 may infer that the request is for user performance data for historical brushing sessions, such as average user performance metrics or a comparison of the user's performance in her ten most recent brushing sessions to the user's performance in all of her brushing sessions.

In some embodiments, the grammar mapping module 312 may find synonyms or nicknames for words or phrases in the input to determine the request. For example, for the input, “Set toothbrush to gentle mode,” the grammar mapping module 312 may determine that sensitive is synonymous with gentle, and may identify that the request is to change the brushing mode to the sensitive mode.

After the natural language processing server 302 determines the request, the grammar mapping module 312 may transmit the request to the device from which the voice input was received (e.g., the charging station 104 or the electric toothbrush 102).

The client computing device 310 may be a tablet computer, a cell phone, a personal digital assistant (PDA), a smartphone, a laptop computer, a desktop computer, a portable media player, a home phone, a pager, a wearable computing device, smart glasses, a smart watch or bracelet, a phablet, another smart device, etc. The client computing device 310 may include one or more processors 322, a memory 324, a communication unit (not shown) to transmit and receive data via long-range and short-range communication networks 300, and a user interface (not shown) for presenting data to the user. The memory 324 may store, for example, instructions for a toothbrush application 326 that receives electric toothbrush data and user performance data related to the user's brushing performance from the electric toothbrush 102 or the charging station 104 via a short-range communication link, such as Bluetooth™. The toothbrush application 326 may then analyze the electric toothbrush data and/or user performance data to identify electric toothbrush and user performance metrics, for example, and may present the user performance metrics on the user interface. User performance metrics may include for example, a proportion of the total surface area covered by the user in the most recent brushing session, and the average amount of force exerted on the teeth during the most recent brushing session.

In some embodiments, the toothbrush application 326 transmits the electric toothbrush data and/or user performance data to a toothbrush server which analyzes the electric toothbrush data and/or user performance data and provides electric toothbrush and user performance metrics to the toothbrush application 326 for display on the user interface. Also in some embodiments, the toothbrush application 326 or the toothbrush server stores the electric toothbrush and user performance metrics as historical data which may be used to compare to current electric toothbrush and user performance metrics. For example, the historical data may be used to train a machine learning model to identify the user based on the user's performance metrics or to predict the user's performance metrics using the machine learning model and determine whether the user has outperformed or underperformed predicted user performance metrics in the user's current brushing session.

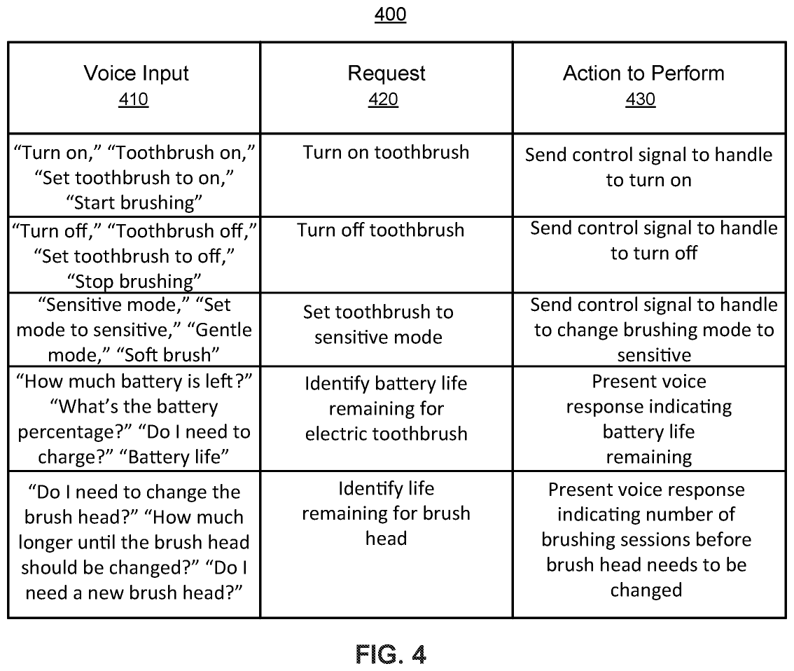

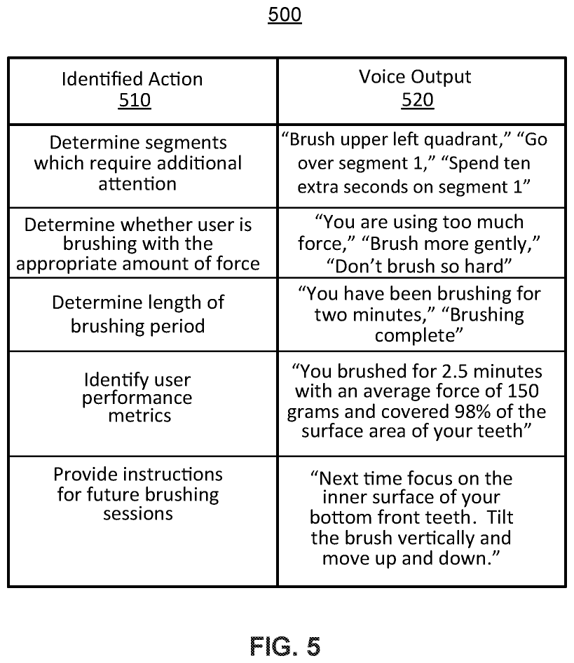

FIG. 4 provides example requests which may be identified from the user's voice input and example actions for the voice assistant application 350 to perform based on the requests. In some embodiments, the voice assistant application 350 provides a voice output which is not in response to a request. For example, at the beginning of a brushing session, the voice assistant application 350 may provide voice output requesting the user to identify herself, so that the voice assistant application 350 may retrieve data for the user from a user profile, such as previous requests made by the user, historical user performance data for the user, machine learning models generated for the user trained using the user's historical user performance data, etc. Accordingly, the voice assistant application 350 may provide voice output that is specific to the identified user, such as voice output that includes the user's name, voice output indicative of the identifier user's performance metrics or historical performance data, etc. Other examples may include voice output instructing the user to charge the electric toothbrush 102 or change the electric toothbrush head 90 when the voice assistant application 350 determines that it is necessary to do so, regardless of whether the user requested this information. FIG. 5 provides example actions that the voice assistant application 350 may take automatically without first receiving a request from the user, and examples of the resulting voice output provided by the voice assistant application 350.

FIG. 4 illustrates an example table 400 having example voice inputs 410 that may be provided to the voice assistant application 350, and example requests 420 and actions 430 for the voice assistant application 350 to perform based on the received voice inputs 410. The example requests 420 and actions to perform 430 may be stored in a database of candidate requests and corresponding actions. Furthermore, a set of steps may be stored in the database for carrying out each action. The database may be communicatively coupled to the electric toothbrush 102, the charging station 104, and/or the action determination server 304.

The example voice inputs 410 may not be pre-stored voice inputs and instead the voice assistant application 350 may identify a corresponding request from a voice input using the speech recognition module 338, the speech recognition server, and/or the natural language processing server 302. A grammar module 312 included in the voice assistant application 350 or the natural language processing server 302 may obtain a set of candidate requests from the database. The grammar module 312 may then assign a probability to each candidate request based on the likelihood that the candidate request corresponds to the voice input. In some embodiments, the candidate requests may be ranked based on their respective probabilities, and the candidate request having the highest probability may be identified as the request. For example, when the voice input includes the word “battery,” the grammar module 312 may determine that candidate requests related to the electric toothbrush head 90, the brushing mode, and the user's brushing performance are unlikely to correspond to the voice input, and may assign low probabilities to these candidate requests.

If the grammar module 312 cannot determine a request based on the text input or determines a request having a likelihood which is less than a predetermined likelihood threshold, the grammar module 312 may cause the voice assistant application 350 to provide follow up questions to the user for additional input.

In any event, the grammar module 312 may determine that the corresponding request for voice input such as, “Turn on,” “Toothbrush on,” “Set toothbrush to on,” and “Start brushing,” is to turn on the electric toothbrush 420. The grammar module 312 may determine that the corresponding request for voice input such as, “Turn off,” “Toothbrush off,” “Set toothbrush to off,” and “Stop brushing,” is to turn off the electric toothbrush 420. Furthermore, the grammar module 312 may determine that the corresponding request for voice input such as, “Sensitive mode,” “Set mode to sensitive,” “Gentle mode,” and “Soft brush,” is to set the electric toothbrush to the sensitive mode. Additionally, the grammar module 312 may determine that the corresponding request for voice input such as, “How much battery is left?” “What's the battery percentage?” “Do I need to charge?” and “Battery life,” is to identify the battery life remaining for the electric toothbrush 102. Still further, the grammar module 312 may determine that the corresponding request for voice input such as, “Do I need to change the brush head?” “How much longer until the brush head should be changed?” and “Do I need a new brush head?” is to identify the life remaining for the electric toothbrush head 90.

In some embodiments, the grammar module 312 may identify a request based on a particular term or phrase included in the voice input and may filter the remaining terms or phrases from the analysis. For example, the grammar module 312 may identify the request is to turn on the toothbrush based on the phrase, “Toothbrush on,” and may filter remaining terms such as, “now” and “please” from the analysis.

When the voice assistant 350 determines the request based on the voice input for example, via the grammar module 312, the voice assistant 350 may identify an action to perform in response to the request and/or one or more steps to take to carry out the requested action. As mentioned above, the voice assistant application 350 may identify an action to perform using the action determination module 340 and/or the action determination server 304. For example, the action determination module 340 and/or the action determination server 304 may obtain an action corresponding to the request and/or one or more steps to take to carry out the requested action from the database.

As shown in the example table 400, the corresponding action 430 for the request 420 to turn the toothbrush on is to send a control signal to the electric toothbrush 102, and more specifically, the electric toothbrush handle 35 to turn on the electric toothbrush 102. This action may require one step of sending the control signal. The corresponding action 430 for the request 420 to turn the toothbrush off is to send a control signal to the electric toothbrush 102, and more specifically, the electric toothbrush handle 35 to turn off the electric toothbrush 102. This action may also require one step of sending the control signal. Additionally, the corresponding action 430 for the request 420 to set the electric toothbrush 102 to the sensitive mode is to send a control signal to the electric toothbrush 102, and more specifically, the electric toothbrush handle 35 to change the brushing mode to sensitive. Once again, this action may require one step of sending the control signal.

Moreover, the corresponding action 430 for the request 420 to identify the battery life remaining for the electric toothbrush 102 is to present a voice response indicating the battery life remaining. This action may require multiple steps, including a first step to obtain electric toothbrush data such as battery life data from the electric toothbrush 102 via a short-range communication, by for example sending a request to the electric toothbrush 102 for the battery life data. The action may also include a second step of generating and presenting a voice response indicating the battery life remaining based on one or more characteristics of the electric toothbrush, such as the received battery life data.

Furthermore, the corresponding action 430 for the request 420 to identify the life remaining for the electric toothbrush head 90 is to present a voice response indicating the number of brushing sessions before the electric toothbrush head 90 needs to be changed. This action may require multiple steps, including a first step to obtain electric toothbrush data, such as the number of brushing sessions or the amount of time in which the electric toothbrush head 90 has been used for example, from the client computing device 310. The action may also include a second step of obtaining historical data indicating the average number of brushing sessions before the user changes the electric toothbrush head 90. The historical data may also be obtained from the client computing device 310. Still further, the action may include a third step of obtaining user performance metrics related to the amount of force exerted when using the electric toothbrush head 90, such an average amount of force, a maximum amount of force, etc.

A machine learning model may also be obtained for estimating the number of brushing sessions remaining before the electric toothbrush head 90 needs to be changed based on the number of brushing sessions in which the electric toothbrush head 90 has been used, the historical data indicating the average number of brushing sessions before the user changes the electric toothbrush head 90, and the user performance metrics related to the amount of force exerted when using the electric toothbrush head 90. The action may also include a fourth step of applying the number of brushing sessions in which the electric toothbrush head 90 has been used, the historical data indicating the average number of brushing sessions before the user changes the electric toothbrush head 90, and the user performance metrics related to the amount of force exerted when using the electric toothbrush head 90 to the machine learning model to identify one or more characteristics of the electric toothbrush, such as the life remaining for the electric toothbrush head 90. Alternatively, the fourth step may be to subtract the number of brushing sessions in which the electric toothbrush head 90 has been used from a predetermined or calculated total number of brushing sessions for the electric toothbrush head 90 before the electric toothbrush head 90 needs to be changed. Moreover, the action may include a fifth step of generating and presenting a voice response indicating the number of brushing sessions before the electric toothbrush head 90 needs to be changed.

The requests 420 included in the table 400 are merely a few example requests 420 for ease of illustration only. The voice assistant application 350 may obtain any suitable number of requests related to the electric toothbrush 102. Moreover, while the database may initially include a predetermined number of candidate requests, additional requests may be provided to the database as candidate requests. For example, additional requests may be learned based on the user's response to follow up questions from the voice assistant application 350. For example, if the voice input is, “Whiten my teeth, please,” the voice assistant application 350 may learn, based on the user's response to follow up questions, that the request is a combination of a first request to turn on the electric toothbrush 102 and a second request to set the electric toothbrush 102 to the whitening mode.

FIG. 5 illustrates an example table 500 having example actions 510 that may be identified by the voice assistant application 350, and example voice outputs 520 for the voice assistant application 350 to present based on the identified actions 510. The example actions 510 may be stored in a database of actions. Furthermore, a set of steps may be stored in the database for carrying out each action. The database may be communicatively coupled to the electric toothbrush 102, the charging station 104, and/or the action determination server 304.

In some embodiments, the actions 510 are automatically identified by the voice assistant application 350 and performed regardless of whether the user provides a request. For example, in some scenarios, the voice assistant application 350 automatically identifies segments of the user's teeth which require additional attention at the end of each brushing session and presents voice output to the user indicating the identified segments. In another example, the voice assistant application 350 may automatically identify and present user performance metrics to the user at the end of each brushing session. In yet another example, the voice assistant application 350 may automatically adjust the volume of the speaker 108 based on the noise level for the area surrounding the electric toothbrush 102 or delay the voice output provided via the speaker 108. The microphone 106 may be used to detect the noise level. When the noise level exceeds a threshold noise level for example, based on noise coming from the electric toothbrush 102, the voice assistant 350 may increase the volume of the speaker 108. Then when the noise level drops below the threshold noise level, the voice assistant may decrease the volume of the speaker 108. In other embodiments, the actions 510 are identified and performed in response to a request, as in the example table 400 shown in FIG. 4.

As shown in the example table 500, example voice output 520 corresponding to the action of determining segments in the user's teeth which require additional attention may include, “Brush upper left quadrant,” “Go over segment 1,” “Spend ten extra seconds on segment 1.” Each segment may have a corresponding numerical indicator, and the voice output may include the numerical indicator corresponding to the segment rather than a description of the segment, such as the upper left quadrant or the chewing surface of the upper left quadrant. This action may require several steps, including a first step to obtain sensor data from the electric toothbrush 102 indicating the positions of the electric toothbrush 102 at several instances in time, for example from multi-axis accelerometers and/or cameras included in the electric toothbrush 102. The sensor data may also include data indicating the amount of force exerted by the user at several instances in time, for example from pressure sensors included in the electric toothbrush 102.

The second step may be to analyze the positions at several instances in time to identify movement of the electric toothbrush 102 and the amount of force exerted at each position to identify segments of the user's teeth which have not been brushed at all or have not been brushed with a threshold amount of force. A third step may be to identify for each segment, the proportion of the total surface area that has been brushed. Furthermore, the action may include a fourth step of obtaining historical user performance data for the user to identify segments which have not been brushed as thoroughly as other segments in the past. The historical user performance data may be obtained from the client computing device 310 via the toothbrush application 326. Then in a fifth step, the voice assistant application 350 may determine the segments which require additional attention by comparing the proportion of the total surface area that has been brushed for a segment to a threshold amount (e.g., 90 percent), identifying segments of the user's teeth which have not been brushed at all or have not been brushed with a threshold amount of force, and/or identifying segments from the historical user performance data which have not been brushed as thoroughly as other segments in the past. Moreover, the action may include a sixth step of generating and presenting the voice output indicating the segments which require additional attention.

Example voice output 520 corresponding to the action of whether the user is brushing with the appropriate amount of force may include, “You are using too much force,” “Brush more gently,” and “Don't brush so hard.” This action may require several steps, including a first step to obtain sensor data from the electric toothbrush 102 indicating the force exerted, such as the average amount of force exerted during the brushing session, the maximum amount of force exerted, etc. In a second step, the voice assistant application 350 may compare the force to a brushing force threshold (e.g., 100 grams) and may generate and present voice output telling the user to increase or decrease the amount of force based on the comparison. In some embodiments, if the user is within a threshold variance (e.g., 50 grams) of the brushing force threshold, the voice assistant 350 may not generate voice output, or the voice output may indicate that the user is brushing with the appropriate amount of force. If the user is using more force than the summation of the brushing force threshold and the threshold variance, the voice assistant 350 may generate voice output instructing the user to decrease the force. If the user is using less than the difference between the brushing force threshold and the threshold variance, the voice assistant 350 may generate voice output instructing the user to increase the force.

Example voice output 520 corresponding to the action of determining the length of the brushing session includes, “You have been brushing for two minutes,” and “Brushing complete.” This action may include two steps of obtaining the length of the brushing session from the electric toothbrush 102 and generating and presenting voice output indicating the obtained length.

Example voice output 520 corresponding to the action of identifying user performance metrics for the brushing session includes, “You brushed for 2.5 minutes with an average force of 150 grams and covered 98% of the surface area of your teeth.” This action may require several steps, including a first step to obtain sensor data from the electric toothbrush 102 indicating the positions of the electric toothbrush 102 at several instances in time, for example from multi-axis accelerometers and/or cameras included in the electric toothbrush 102. The sensor data may also include data indicating the amount of force exerted by the user at several instances in time, for example from pressure sensors included in the electric toothbrush 102. Moreover, the sensor data may include the amount of time for the brushing session. The second step may be to analyze the positions at several instances in time to identify movement of the electric toothbrush 102 and the amount of force exerted at each position to identify segments of the user's teeth which have not been brushed at all or have not been brushed with a threshold amount of force. In this manner, the voice assistant application 350 may determine the average amount of force exerted during the brushing session and the proportion of the total surface area of the teeth covered during the brushing session. The third step may be to generate and present voice output indicating the amount of time for the brushing session, the average amount of force exerted during the brushing session, and the proportion of the total surface area of the teeth covered during the brushing session.

Example voice output 520 corresponding to the action of providing instructions for future brushing sessions includes, “Next time focus on the inner surface of your bottom front teeth. Tilt the brush vertically and move up and down.” The instructions for future brushing sessions may be identified based on shortcomings from the user's most recent brushing session or shortcomings from historical brushing sessions. Accordingly, to identify these shortcomings, the actions may include determining segments which require additional attention, determining whether the user is brushing with the appropriate amount of force, and determining the length of the brushing period, as described above. Based on these determinations, the voice assistant application 350 may identify areas where the user can improve her brushing habits. The voice assistant application 350 may then generate a voice instruction to the help the user improve in the identified area.

For example, when determining segments which require additional attention, the voice assistant application 350 may determine that the user did not brush a middle portion of the inner surface of the lower left quadrant and has not brushed the middle portion of the inner surface of the lower left quadrant in the previous five brushing sessions without receiving specific instructions from the voice assistant application 350 to do so. Accordingly, the voice assistant application 350 may provide voice instructions to focus on the middle portion of the inner surface of the lower left quadrant, and may provide instructions on how to position the brush to cover the middle portion of the inner surface of the lower left quadrant. In another example, when determining the length of the brushing period, the voice assistant application 350 may determine that the length of the brushing period has decreased by an average of five seconds in each of the previous three brushing sessions. Accordingly, the voice assistant application 350 may provide voice instructions to the user to remember to brush for at least two minutes.

The actions 510 included in the table 500 are merely a few example actions 510 for ease of illustration only. The voice assistant application 350 may perform any suitable number of actions related to the electric toothbrush 102.

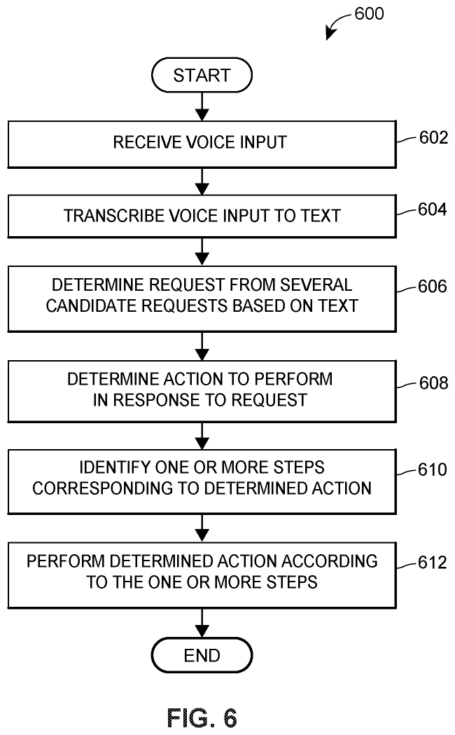

FIG. 6 illustrates a flow diagram representing an example method 600 for providing voice assistance to a user regarding an electric toothbrush. The method 600 may be performed by the voice assistant application 350 and executed on the device storing the voice assistant application 350, such as the charging station 104 or the electric toothbrush 102. In some embodiments, the method 600 may be implemented in a set of instructions stored on a non-transitory computer-readable memory and executable on one or more processors of the charging station 104 or the electric toothbrush 102. For example, the method 600 may be at least partially performed by the speech recognition module 338, the action determination module 340, and the control module 342, as shown in FIG. 3.

At block 602, voice input from the user is received via the microphone(s) 106. The voice input is then transcribed to text input (block 604). For example, the voice assistant application 350 may transcribe the voice input to text input via the speech recognition module 338. In another example, the voice assistant application 350 may provide the raw voice input to a speech recognition server to transcribe the voice input to text input, and may receive the transcribed text input from the speech recognition server.

Then at block 606, a request is determined from several candidate requested based on the transcribed text input. More specifically, the text input may be compared to grammar rules stored by the voice assistant application 350, or may be transmitted to the natural language processing server 302. For example, the voice assistant application 350 or the natural language processing server 302 may store a list of candidate requests that the voice assistant application 350 can handle, such as turning on and off the electric toothbrush, selecting the brushing mode for the electric toothbrush, identifying the battery life remaining for the electric toothbrush 102, identifying the life remaining for the brush head 90, identifying user performance metrics for the current brushing session or previous brushing sessions, sending user performance data to the user's client computing device 310, etc.

A grammar mapping module 312 may then compare the text input to grammar rules in a grammar rules database 314. Moreover, the grammar mapping module 112 may make inferences based on context. In some embodiments, the grammar mapping module 312 may find synonyms or nicknames for words or phrases in the input to determine the request. Using the grammar rules, inferences, synonyms, and nicknames, the grammar module 312 may assign a probability to each candidate request based on the likelihood that the candidate request corresponds to the text input. In some embodiments, the candidate requests may be ranked based on their respective probabilities, and the candidate request having the highest probability may be identified as the request.

At block 608, the voice assistant application 350 determines an action to perform in response to the request. The candidate requests and corresponding actions to perform may be may be stored in a database. Furthermore, a set of steps for carrying out each action may be stored in the database. When the voice assistant 350 determines the request, the voice assistant 350 may identify an action to perform via the action determination module 340 or by providing the request to the action determination server 304. For example, the action determination module 340 and/or the action determination server 304 may obtain an action corresponding to the request and/or one or more steps to take to carry out the requested action from the database (block 610). The one or more steps may include receiving sensor data from the electric toothbrush 102, receiving data from the user's client computing device 310, providing voice output to the user responding to the request, providing a visual indicator such as light from an LED to the user responding to the request, and/or sending a control signal to the electric toothbrush 102 to control operation of the electric toothbrush 102 based on the request. The visual indicator may be used to indicate for example, that the electric toothbrush 102 has been turned on or turned off in response to a request by the user to turn on or turn off electric toothbrush 102. In some embodiments, the electric toothbrush 102 may include one or more LEDs which may be controlled by the voice assistant application 350. The LEDs may be used to indicate whether the electric toothbrush 102 is turned on or turned off, the mode for the electric toothbrush 102, such as daily clean, massage or gum care, sensitive, whitening, deep clean, or tongue clean, the brush speed or frequency for the electric toothbrush head 90, etc. More specifically, in one example, the voice assistant application 350 may send a control signal to a first LED to turn on the first LED indicating that the electric toothbrush 102 has been turned on. In another example, the voice assistant application 350 may send a control signal to a series of LEDs to turn on the series of LEDs indicating that the electric toothbrush 102 is in the whitening mode. The one or more steps may also include providing data, such as user performance data indicative of the user's brushing behavior to the client computing device 310 for presentation or storage at an electric toothbrush application 326 executing on the client computing device 310.

Then at block 612, the voice assistant application 350 performs the determined action according to the one or more steps to carry out the action. As described above, the voice assistant application 350 may provide voice output to the user, via the speaker(s) 108, responding to the request or may send a control signal to the electric toothbrush 102 to control operation of the electric toothbrush 102 based on the request.

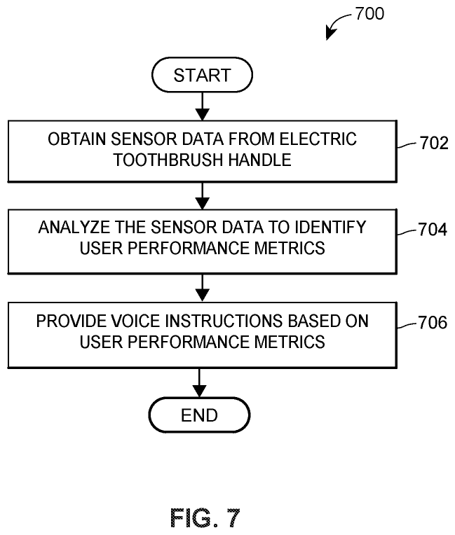

FIG. 7 illustrates a flow diagram representing another example method 700 for providing voice assistance to a user regarding an electric toothbrush. The method 700 may be performed by the voice assistant application 350 and executed on the device storing the voice assistant application 350, such as the charging station 104 or the electric toothbrush 102. In some embodiments, the method 700 may be implemented in a set of instructions stored on a non-transitory computer-readable memory and executable on one or more processors of the charging station 104 or the electric toothbrush 102. For example, the method 700 may be at least partially performed by the action determination module 340, and the control module 342 as shown in FIG. 3.

In the example method 700 the voice output is automatically provided without first receiving a request from the user. At block 702, sensor data is obtained from the electric toothbrush 102 during the current brushing session, such as from the electric toothbrush handle 35. Sensor data may also be obtained from the user's client computing device 310, such as historical sensor data or historical user performance data. The sensor data may include data indicating the positions of the electric toothbrush 102 at several instances in time, for example from multi-axis accelerometers and/or cameras included in the electric toothbrush 102. The sensor data may also include data indicating the amount of force exerted by the user at several instances in time, for example from pressure sensors included in the electric toothbrush 102. Moreover, the sensor data may include the amount of time for the brushing session.

Then at block 704, the sensor data is analyzed to determine user performance metrics. The user performance metrics may include the amount of time for the brushing session, the average amount of force exerted during the brushing session, the proportion of the total surface area of the teeth covered during the brushing session, the number of segments which have not been brushed at all or have not been brushed with a threshold amount of force, etc. The user performance metrics may also include comparative metrics based on the user's historical performance metrics. For example, the comparative metrics may include a difference between the amount of time for the brushing session and the average amount of time for the user's historical brushing sessions. The comparative metrics may also include a difference in the proportion of the total surface area of the teeth covered during the brushing session and the average proportion of the total surface area of the teeth covered during the user's historical brushing sessions.

At block 706, the voice assistant application 350 provides voice instructions, via the speaker(s) 108, in accordance with the user performance metrics. For example, the voice instructions may be to use more or less force when brushing or to provide additional attention to a particular segment of the user's teeth. The voice instructions may also be instructions for future brushing sessions based on shortcomings from the user's most recent brushing session or shortcomings from historical brushing sessions.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as exemplary only—and does not describe every possible embodiment, as describing every possible embodiment would be impractical, if not impossible. One could implement numerous alternate embodiments, using either current technology or technology developed after the filing date of this application. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Every document cited herein, including any cross-referenced or related patent or application and any patent application or patent to which this application claims priority or benefit thereof, is hereby incorporated herein by reference in its entirety, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

What is claimed is:

1. A system for providing voice assistance regarding an electric toothbrush, the system comprising:
an electric toothbrush; and
a charging station configured to provide power to the electric toothbrush, the charging station including:
a communication interface;
one or more processors;
a speaker;
a microphone; and
a non-transitory computer-readable memory coupled to the one or more processors, the speaker, the microphone, and the communication interface, and storing thereon instructions that, when executed by the one or more processors, cause the charging station to:
receive, from a user via the microphone, voice input regarding the electric toothbrush; and
provide, to the user via the speaker, voice output related to the electric toothbrush.

2. The system of claim 1, wherein the instructions further cause the charging station to:
analyze the received voice input to determine a request from the user;
obtain electric toothbrush data or user performance data for the electric toothbrush related to the request;
analyze, according to the request, the electric toothbrush data or the user performance data for the electric toothbrush to generate a voice response to the request; and
provide, via the speaker, the voice response to the request.

3. The system of claim 2, wherein the instructions further cause the charging station to adjust operation of the electric toothbrush based on the request.

4. The system of claim 2, wherein to analyze the received voice input to determine a request from the user, the instructions cause the charging station to:
transcribe the voice input into text input;
compare the text input to a set of grammar rules; and
identify a request from a plurality of candidate requests based on the comparison.

5. The system of claim 4, wherein each candidate request is associated with one or more steps for determining the voice response to the candidate request or performing an action related to the electric toothbrush.

6. The system of claim 4, wherein the plurality of candidate requests includes at least one of:
a first candidate request regarding an amount of charge remaining for the electric toothbrush,
a second candidate request regarding an estimated life remaining for an electric toothbrush head removably attached to an electric toothbrush handle,
a third candidate request related to brushing performance of the user,
a fourth candidate request related to a number of brushing sessions remaining before the electric toothbrush requires additional charge,
a fifth candidate request to turn the electric toothbrush on or off, and
a sixth candidate request to change a brushing mode for the electric toothbrush.

7. The system of claim 1, wherein to provide voice output related to the electric toothbrush to the user, the instructions cause the charging station to:
obtain, via the communication interface, sensor data from one or more sensors in the electric toothbrush;
analyze the sensor data to identify one or more user performance metrics related to use of the electric toothbrush; and
provide voice instructions to the user based on the one or more user performance metrics.

8. The system of claim 1, wherein the instructions further cause the charging station to:
obtain an indication of a noise level in an area encompassing the electric toothbrush; and
adjust a volume of the speaker in accordance with the noise level.

9. The system of claim 8, wherein the instructions further cause the charging station to delay the voice output provided via the speaker in accordance with the noise level.

10. The system of claim 1, wherein the electric toothbrush includes an electric toothbrush head removably attached to an electric toothbrush handle, and wherein the instructions further cause the charging station to:
obtain an indication of a number of brushing sessions in which the electric toothbrush head has been used;
determine an estimated life remaining for the electric toothbrush head based on the number of brushing sessions in which the electric toothbrush head has been used; and
provide, via the speaker, the voice output including an indication of the estimated life remaining for the electric toothbrush head.

11. A method for providing voice assistance regarding an electric toothbrush, the method comprising:
receiving, at a charging station that provides power to an electric toothbrush, voice input via a microphone from a user of the electric toothbrush;
analyzing, by the charging station, the received voice input to determine a request from the user;
determining, by the charging station, an action in response to the request; and
performing, by the charging station, the action in response to the request by providing, via a speaker, a voice response to the request, providing a visual indicator, or adjusting operation of the electric toothbrush based on the request.

12. The method of claim 11, wherein performing the action in response to the request further includes transmitting, by one or more processors, information in response to the request to a client device of the user.

13. The method of claim 11, wherein determining an action in response to the request includes determining one or more steps to perform to carry out the action.

14. The method of claim 13, wherein determining one or more steps to perform to carry out the action includes:
obtaining electric toothbrush data for the electric toothbrush;
analyzing the electric toothbrush data to identify one or more characteristics of the electric toothbrush; and
providing voice instructions to the user based on the identified one or more characteristics.

15. The method of claim 11, wherein analyzing the received voice input to determine a request from the user includes:
transcribing the voice input into text input;
comparing the text input to a set of grammar rules; and
identifying a request from a plurality of candidate requests based on the comparison.

16. A method for providing voice assistance regarding an electric toothbrush, the method comprising:
during a brushing session by a user:
obtaining, at a charging station providing power to an electric toothbrush, sensor data from one or more sensors included the electric toothbrush;
analyzing, by the charging station, the sensor data to identify one or more user performance metrics related to use of the electric toothbrush by the user; and
providing, by the charging station via a speaker, voice output to the user based on the one or more user performance metrics.

17. The method of claim 16, further comprising:
obtaining an indication of a noise level in an area encompassing the electric toothbrush; and
adjusting a volume of the speaker in accordance with the noise level or delaying the voice output provided via the speaker.

18. The method of claim 17, further comprising delaying the voice output provided via the speaker in accordance with the noise level.

19. The method of claim 16, wherein analyzing the sensor data to identify one or more user performance metrics includes analyzing the sensor data to identify segments of a set of teeth of the user which have not been brushed during the brushing session, and wherein providing voice output to the user includes providing voice instructions to brush the identified segments.

20. The method of claim 16, wherein analyzing the sensor data to identify one or more user performance metrics includes analyzing the sensor data to determine an estimated life remaining for an electric toothbrush head removably attached to an electric toothbrush handle and determine that the electric toothbrush head removably needs to be changed based on the estimated life remaining, and wherein providing voice output to the user includes providing voice instructions to change the electric toothbrush head.

21. The method of claim 16, wherein analyzing the sensor data to identify one or more user performance metrics includes analyzing the sensor data to determine whether a brushing force used by the user is above or below a brushing force threshold, and wherein providing voice output to the user includes providing voice instructions to increase or decrease the brushing force.

22. The method of claim 16, wherein analyzing the sensor data to identify one or more user performance metrics includes analyzing the sensor data to identify the user, and wherein providing voice output includes providing voice output specific to the identified user.
